SEQUENCE LISTING (1 sequence)

SEQ ID NO: 1 - protein, 6 aa, Artificial Sequence
  Cell Penetrating Peptide
RRRRRR

(19) United States
(12) Patent Application Publication
(10) Pub. No.: US 2018/0200340 A1
(43) Pub. Date: Jul. 19, 2018

(54) Wound Treatment

(71) Applicant: THE UNIVERSITY OF BIRMINGHAM, West Midlands (GB)

(72) Inventors: Liam GROVER, West Midlands (GB); Ann LOGAN, West Midlands (GB); Felicity Jane DE COGAN, West Midlands (GB); Gurpreet CHOUHAN, West Midlands (GB)

(21) Appl. No.: 15/744,164

(22) PCT Filed: Jul. 18, 2016
(86) PCT No.: PCT/GB2016/052166

§ 371 Date: Jan. 12, 2018

(30) Foreign Application Priority Data
Jul. 17, 2015 (GB) 1512582.6

(51) Int. Cl. A61K 38/39; A61K 38/36; A61K 38/17; A61K 47/36; A61K 47/64; A61K 9/70; A61K 9/00; A61P 17/02

(52) U.S. Cl. CPC A61K 38/39; A61K 38/363; A61K 38/1709; A61P 17/02; A61K 47/64; A61K 9/7007; A61K 9/0048; A61K 47/36

(57) ABSTRACT

The application discloses hydrogel wound treatment formulation containing collagen and decorin, and optionally fibrin. This is especially useful for the treatment of the eye. Wound dressings comprising the formulation are also provided, for example in the form of a gellan sheet.

The invention relates to wound treatment formulations comprising hydrogels, the hydrogels containing decorin and collagen.

The treatment formulations may be used, for example, to reduce inflammation, neovascularisation and/or scarring in wounds or sites of tissue disease/damage, such as wounds in the eye.

The use of wound dressings is generally known in the art. A particular problem associated with many wounds, such as those from ophthalmic surgery, infections or burns, is that such wounds often produce scarring. Scars are typically areas of fibrous tissue (fibrosis) that replace normal skin after injury. Scar tissue typically comprises a range of extra-cellular matrix molecules including collagen, but the fibre composition of the matrices is often different to that of normal tissue. Instead of the structural alignment of fibres found in normal tissue, in fibrosis the fibres cross-link in a random basket weave formation. This collagenous scar tissue alignment is usually of inferior functional quality to normal fibre alignment found in normal tissue. This means that the wound, when it heals, often has reduced or impaired mechanical and optical properties, as well as having an adverse visual appearance.

Wound healing itself is complex and involves a number of pathways which leave the tissue healed but sometimes scarred. Scarring is particularly problematic in the eye where it can reduce vision after trauma to the eye due to accidents, infections, disease or eye surgery

Transforming Growth Factor β (TGFβ) plays a role in controlling the immune system, inflammatory responses and their consequences, for example, scarring. TGFβ has three isoforms (TGFβ-1, TGFβ-2 and TGFβ-3), having highly conserved regions but several divergent regions. TGFβ-1 has been localised in brain, eye, cartilage, bone and skin, suggesting a role in the differentiation of these tissues. TGFβ-2 is expressed in neurons and astroglial cells of the CNS and also appears to be involved in tumour development. Both TGFβ-1 and TGFβ-2 have been shown to be potent stimulators of scar formation. TGFβ-3 appears to be involved in normal palate and lung morphogenesis (Kubiczkove L. et al, J. Medicine (2012) 10, 183) and may act as an anti-fibrotic agent. Removing TGFβ-1 and -2 from wounds, or changing the isoform balance, reduces scarring.

Decorin is a glycoprotein of on average 90-140 kD molecular weight. It belongs to the small-leucine rich proteoglycan (SLRP) family and consists of a protein core containing leucine repeats with a glucosaminoglycan (GAG) chain consisting of either chondroitin sulphate or dermatan sulphate. It binds to type I collagen fibrils through the decorin type I collagen binding region as shown by Kalamajski et al (J. Biol. Chem (2007) 286(22) 16062-16067). Decorin has been found to either enhance or inhibit the activity of TGF-β isoforms-1 and -2, as well as other growth factors involved in the wound response. Its use as a wound treatment agent is shown in U.S. Pat. No. 5,510,328.

Decorin acts as a TGF β-1/2 antagonist and reduces scarring. Reports show that in acute scarring the dominant effect of Decorin is anti-fibrogenic through suppression of inflammatory fibrosis by neutralisation of TGF β-1/2. Decorin also binds directly to collagen and one of its functions is to influence on the organisation of collagen during wound healing. When bound to collagen the inventors believe that the TGFβ binding sites on Decorin are more effectively presented for optimal antagonistic activity.

Logan A et al (Experimental Neurology (1999) 159, 504-510) describe Decorin-induced inhibition of scarring in a model of cerebral lesion. Botfield et al (Brain (2013) 136, 2842-2858) demonstrate the prevention of scarring by Decorin in a model of hydrocephalus. Ahmed Z et al (Neurobiology of Disease (2014) 64, 163-176) describes the blocking of scarring and cystic cavitation with Decorin in chronic spinal cord wounds. Hill et al (Investigative Ophthalmology and Vision Science (2015) 56, 3743-3757) show that Decorin induces fibrolysis of existing trabecular meshwork scars in a glaucoma model.

A range of cellulose based membranes, gums and polymer gels have been described for the delivery of drugs and cell delivery systems to wounds (see US 2012/0231038, WO 2013/079605 and WO 2014/140549). The inventors have realised that by combining the properties of decorin and collagen so that, for example, decorin is bound to the collagen, it would be possible to apply decorin to the wound in a form that optimises its ability to bind TGFβ and other regulatory factors, such as VEGF, PDGF, EGF etc. The decorin and collagen combination within the topical formulation would more effectively absorb and remove TGF-β/other factors than would decorin when presented alone, resulting in enhanced anti-scarring and other bioactivities. Moreover, placing the combination in a fluid gel, when associated with cell penetrating peptides (CPP), decorin and collagen could be placed in contact with, for example, the surface of the eye for periods typically exceeding four hours. This is better than traditional formulations which typically use aqueous eye drops which do not remain in contact with the eye for very long before they are rapidly blinked away with tears. When bound to the decorin-collagen complex, TGFβ and other bound factors are more effectively sequestered so that they cannot signal fibrosis/inflammation/angiogenesis, and are then removed from the eye surface as the fluid hydrogel is slowly blinked away.

Fibrin, factor Ia, may also be used instead of or in addition to collagen. Fibrin is known to be bound by decorin and modulate fibrin assembly and structure.

The invention therefore provides a wound treatment formulation comprising a gel, wherein the gel contains collagen and decorin. Fibrin may be used instead of or in addition to collagen, in combination with decorin. Typically the formulation is a gel, such as a hydrogel. Hydrogels typically are networks of polymer chains that are hydrophilic, and are sometimes found as a gel in which water is the dispersion medium. Examples of hydrogels include gelatin, poly(alkylene oxides) such as poly(ethylene oxide), poly(meth)acrylates and methyl cellulose. Most typically, the hydrogel comprises a gellan, a generally known polysaccharide gum which is produced by Pseudomonas elodea. This is a hydrocolloid manufactured by fermentation from a carbohydrate source. Deacylation is carried out by treating the product with alkali.

Gellan may be used at 0.5%-3% wt/vol of the formulation prior to setting, for example 1-2.5% wt/vol or 2% wt/vol.

Alginate may also be used.

Mixtures of gellan and alginate may be used, for example at 0.5%-1% gellan to 0.25-0.75% alginate, for example 0.75% gellan and 0.50% wt/wt alginate.

The hydrogel may or may not be present in addition to the collagen or fibrin. The amount of hydrogel may be adjusted to increase or decrease the viscosity of the formulations.

Wound treatment formulations may be used to treat damaged tissue from trauma such as accidental damage or surgery, or damage due to infection or disease.

Gellan gum is based on a linear structure of repeating glucose rhamnose and glucuronic acid units. In high acyl gum, two acyl side chains of acetate and glycerate are present. Both substituents are present on the same glucose molecule and on average there is one glycerate per repeating unit and one acetate every two repeating units. In low acyl gellan gums the acyl groups are removed. High acyl products tend to form soft elastic gels while gellan gum produces firmer, less elastic gels. The gellan gum may be high or low acyl or a combination thereof.

Mixtures of hydrocolloids may be used.

Typically decorin is used at 0.1 mg/ml to 0.5 mg/ml.

Collagen may be used at 0.1 mg/ml to 2 mg/ml, typically 0.2 mg/ml to 1.0 mg/ml or 0.25 mg/ml to 1.0 mg/ml.

Fibrin may be used at similar concentrations instead of or in addition to collagen.

These may be dissolved in or mixed in a pharmaceutically acceptable liquid medium, such as water or saline with, for example, sodium chloride or potassium chloride. One or more preservatives may also be provided.

The collagen and decorin are typically bound together.

The collagen and decorin may be further complexed with a cell penetrating peptide (CPP). CPP may be, for example, a polypeptide of up to 20 amino acids in length, comprising a continuous region of at least 2, more typically at least 4 basic amino acids. Typically the polypeptide forms structures of 2-5,000 nm.

The CPP may have the formula (B)ₙ(A)ₘ, where B is a basic amino acid, A is an acidic amino acid and m and n are integers, where n is at least 4 and m is less than n. Typically the polypeptide consists of basic amino acids, such as arginine, lysine and histidine. The acidic amino acid is typically selected from aspartate and glutamate. This is described in WO2015/114324.

Such CPP include, for example, the sequence RRRRRR. The sequences may or may not be covalently bound to, for example, the decorin, via cross-linking the peptide to decorin via an amine group of the peptide and thiol group of decorin.

Decorin bound to such sequences has been found by the Applicant to be transported across the cornea of the eye into the aqueous humour better than unbound decorin.

Gellan gums are especially useful for the treatment of wounds to the eye. It has been found to be particularly advantageous as it is readily able to incorporate one or more biologically active ingredients, such as the decorin.

Decorins are generally known in the art and are available under the proprietary name “GALACORIN™”. Other preparations of decorin are commercially available.

The formulation may additionally comprise a drug, a matrikine, a profibrotic agent, a pro- or anti-angiogenic agent, an antibody, a pro-/anti-inflammatory agent, an antimicrobial agent, a proteoglycan or an analgesic. Gels may additionally comprise a biologically acceptable buffer.

Collagen may be mature or immature collagen. It is typically selected from type I and type IV collagen, more typically type I collagen or combinations thereof.

The formulation may be used in combination with a wound dressing. The wound dressing may be, for example, made of a cellulosic material. Alternatively, dressings for eye wounds may use amniotic membrane. An alternative to the amniotic membrane is to use a sheet of gellan gum. The gellan gum may be, for example, 0.5 to 5 mm thick and may comprise a cross linking agent. It may also comprise a polymer, such as gelatin or polyvinylacelate in a ratio of 90% to 50:50 weight % gellan:polymer. Such sheets are typically made by heating 0.05 to 5% w/w, especially 0.05 to 2% w/w gellan in an aqueous liquid to form liquid gellan and cooling to form a sheet. Biologically active compounds such as decorin may be incorporated into the sheet.

The formulation of the invention may be used between the wound and the wound dressing. It may assist in adhering the wound dressing to the wound.

The invention will now be described by way of example only with reference to the following figures:

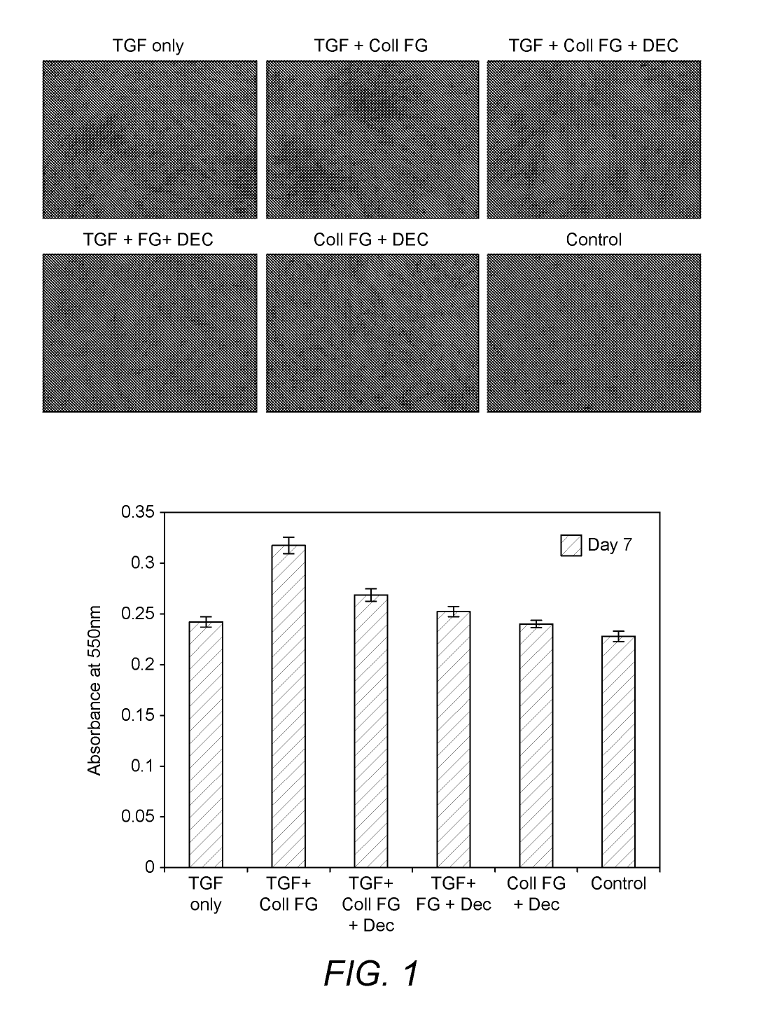

FIG. 1: The addition of soluble collagen to the gellan fluid gel increases the potency of decorin, whereby a reduction in the extracellular matrix collagen production is noted in Human Dermal Fibroblast cultures.

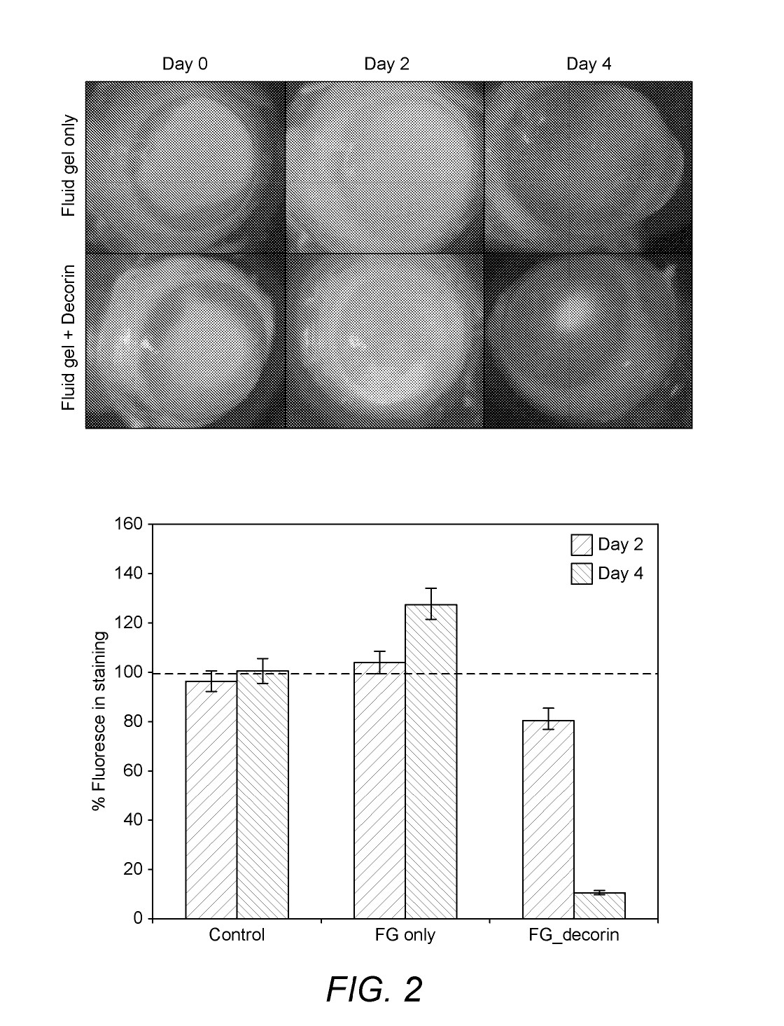

FIG. 2: Images illustrating re-epithelialisation in epithelia abraded rat eyes, when treated with fluid gels (FG) with and without decorin (a). Fluorescein staining reduced in rat eyes treated with the decorin fluid gel over 4 days, when compared to fluid gels without decorin in the ex vivo (organ culture)model (b).

FIG. 1 shows that collagen increases the potency of decorin, whereby collagen deposition was reduced in human dermal fibroblast culture. Gels were prepared with 2% w/v gellan, TGF-beta 10 ng/ml, collagen 3 mg/ml, decorin 0.24 mg/ml, in water.

The formulation can be dropped or sprayed to occlude the wound surface.

FIG. 2 illustrates the re-epithelialisation of the corneal surface when the gellan containing formulation is dropped onto the surface of the eye. Fluorescein staining was reduced in eyes treated with the gels containing decorin.

This shows that the combination of decorin and collagen has advantageous properties. The inventors have also found that the wound treatment can be sprayed to form a thin and even coating on a surface. Cell-loaded gels demonstrate an even spreading of the gel as well as maintaining cell viability over several days.

1. A hydrogel wound treatment formulation comprising collagen and fibrin, or collagen and decorin, and optionally fibrin; where the hydrogel is a fluid gel.

2. (canceled)

3. The wound treatment formulation of claim 1 wherein the hydrogel comprises gellan.

4. The wound treatment formulation of claim 1 wherein the collagen and decorin are complexed with a cell penetrating peptide

5. The wound treatment formulation of claim 1 wherein the collagen is a type I collagen.

6. The wound treatment formulation of claim 1 wherein the decorin is bound to collagen.

7. A method of reducing scarring, the method comprising applying the formulation of claim 1 to a wound or site of infection or disease.

8. The method of claim 7 wherein the wound or site of infection or disease is in or on an eye.

9. The wound treatment formulation of claim 1 adapted for use in the treatment of a wound or site of infection or disease.

10. The wound treatment formulation of claim 1 adapted for use in the treatment of an eye.

11. The wound treatment formulation of claim 1 in the form of a wound dressing.

12. The wound treatment formulation of claim 11 comprising a gellan sheet.

13. The wound treatment formulation of claim 1 wherein the collagen is complexed with a cell penetrating peptide.

14. The method of claim 7 wherein the hydrogel comprises gellan.

15. The method of claim 7 wherein the collagen is complexed with a cell penetrating peptide.

16. The method of claim 7 wherein the collagen and decorin are complexed with a cell penetrating peptide

17. The method of claim 7 wherein the collagen is a type I collagen.

18. The method of claim 7 wherein the decorin is bound to collagen.
